(19) United States
(12) Patent Application Publication
(10) Pub. No.: US 2014/0257210 A1
(43) Pub. Date: Sep. 11, 2014

(54) Wound Management Method and Apparatus

(71) Applicant: Arnold R. Leiboff, Stony Brook, NY (US)

(72) Inventor: Arnold R. Leiboff, Stony Brook, NY (US)

(21) Appl. No.: 14/180,492

(22) Filed: Feb. 14, 2014

Related U.S. Application Data
(60) Provisional application No. 61/766,751, filed on Feb. 20, 2013.

(51) Int. Cl. A61F 13/00; A61M 1/00

(52) U.S. Cl. CPC A61F 13/00021; A61M 1/0088
USPC 604/319

(57) ABSTRACT

A method for managing a closed incisional wound that includes at least one elastomeric drain inserted partially into the wound, an open cell foam sponge in flow communication with the drains and wound, and to which negative pressure is applied by a vacuum source. The negative pressure is transmitted into the otherwise closed wound in order to improve drainage and expedite wound healing.

### CROSS-REFERENCE TO RELATED APPLICATION

This application claims priority of U.S. provisional patent application Ser. No. 61/766,751 filed Feb. 20, 2013, which is incorporated by reference herein.

### FIELD OF THE INVENTION

The present invention relates generally to wound management apparatus and methods for managing a surgical wound after an operation in order to expedite wound healing and reduce the risk of wound complications.

### BACKGROUND OF THE INVENTION

Wound infection remains a major problem in surgery. Measures to reduce the rate of wound infection, and complications such as wound dehiscence, evisceration and hernia that may result from wound infection, include the use of antiseptics, antibiotics, film and physical barriers and the placement of wound packing and drains. Alternatively, the skin and subcutaneous fat incision of a wound may be left open or partially open. This is frequently done when it is believed that the wound was substantially contaminated by microbes during the surgical procedure. Although the latter measure may be effective in reducing wound infection rates, postoperative wound care is cumbersome and convalescence prolonged, requiring more intensive treatment at a higher cost.

Although there is evidence that wound drains reduce the rate of wound infection, present methods and apparatus for draining wounds are only partially effective, and the rate of wound infections with these methods and apparatus remains substantial.

Negative pressure wound therapy on open wounds promotes perfusion, reduces swelling, reduces granulation tissue formation by facilitating cell migration and cell proliferation, draws wound edges together and removes exudate and infectious materials. New systems to apply negative pressure wound therapy to closed surgical wounds have been developed and have been shown to reduce rates of wound infection. These systems comprise suction pumps and dressings that provide negative pressure to the surface of a wound, but provide no means to deliver negative pressure directly to the interior of a wound. Reported rates of wound infection when these systems are used are still substantial.

A method of managing a closed surgical wound that provides effective drainage and results in a lower rate of wound complications would be highly advantageous, reducing surgical morbidity and the cost of surgical care.

### OBJECTS OF THE INVENTION

A wound management system in accordance with this invention includes a single or multiple soft and flexible elastomeric drains that are secured in an otherwise closed incisional wound (i.e., a surgical incision which, after completion of a surgical operation, has been closed by suturing or stapling the skin edges together) in such a manner that the drains penetrate through the skin incision so that an end of each of the drains remain external to the body. Each drain may be secured to the skin with at least one stitch that runs through the skin and drain. A sponge may be placed over the drains and wound, and covered and secured with an occlusive adhesive film drape that is secured to the skin surrounding the sponge and wound. Suction may be applied through a tube in flow continuity with the sponge, drains and wound to transmit negative pressure through the wound and to aspirate fluid from the wound into a collection container.

There are multiple possible configurations for the drains. A preferred embodiment is a flat channel drain. The surgeon may be provided with multiple drains of particular lengths, or with a single long drain that during a surgical operation, he may cut into drains of shorter length that are suitable for the depth of the wound.

Alternately, a substantially round fluted drain may be used in a similar manner. This fluted structure is seen in round Blake surgical drains.

Alternately, an elastomeric tube with perforations along its length may be used. This structure may be found in Jackson Pratt surgical drains.

Alternatively, a soft rubber tube, such as a Penrose drain, may be used.

To facilitate the performance of the method of this invention, an incisional wound dressing kit may be provided containing the components necessary to dress a wound in accordance with this method, including drains, an open cell foam sponge, strips and sheets of adhesive film drapes with liners (paper or plastic strips or sheets that are attached to the adhesive film and removed from the adhesive film during application of the adhesive film drape and which facilitate the handling and application of the drapes), and a tube assembly comprising a tube, hub, adhesive film, connector and tubing clamp.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 is a top view showing position of drains for use in a method for managing a surgical wound in accordance with this invention.

FIG. 2 is a longitudinal sectional view of the use of apparatus in a method for managing a surgical wound in accordance with this invention.

FIG. 3 is a transverse sectional view of the apparatus for use in a method for managing a surgical wound in accordance with this invention.

FIG. 4A is an end view of a first and preferred embodiment of a drain for use in a method for managing a surgical wound in accordance with this invention.

FIG. 4B is a top view of a first and preferred embodiment of a drain for use in a method for managing a surgical wound in accordance with this invention.

FIG. 4C is a side view of a first and preferred embodiment of a drain for use in a method for managing a surgical wound in accordance with this invention.

FIG. 4D is a perspective view of a first and preferred embodiment of a drain for use in a method for managing a surgical wound in accordance with this invention.

FIG. 5A is an end view of a second embodiment of a drain for use in a method for managing a surgical wound in accordance with this invention.

FIG. 5B is a top view of the second embodiment of a drain for use in a method for managing a surgical wound in accordance with this invention.

FIG. 5C is a side view of a second embodiment of a drain for use in a method for managing a surgical wound in accordance with this invention.

FIG. 5D is a perspective view of a second embodiment of a drain for use in a method for managing a surgical wound in accordance with this invention.

FIG. 6A is an end view of a third embodiment of a drain for use in a method for managing a surgical wound in accordance with this invention.

FIG. 6B is a top view of a third embodiment of a drain for use in a method for managing a surgical wound in accordance with this invention.

FIG. 6C is a side view of a third embodiment of a drain for use in a method for managing a surgical wound in accordance with this invention.

FIG. 6D is a perspective view of a third embodiment of a drain for use in a method for managing a surgical wound in accordance with this invention.

FIG. 7A is an end view of a fourth embodiment of a drain for use in a method for managing a surgical wound in accordance with this invention.

FIG. 7B is a top view of a fourth embodiment of a drain for use in a method for managing a surgical wound in accordance with this invention.

FIG. 7C is a side view of a fourth embodiment of a drain for use in a method for managing a surgical wound in accordance with this invention.

FIG. 7D is a perspective view of a fourth embodiment of a drain for use in a method for managing a surgical wound in accordance with this invention.

FIG. 8 is a flow chart showing the basic steps of the method of this invention.

FIG. 9 shows components of a wound dressing kit that would facilitate the performance of the method of wound management according to this invention.

### DETAILED DESCRIPTION OF THE INVENTION

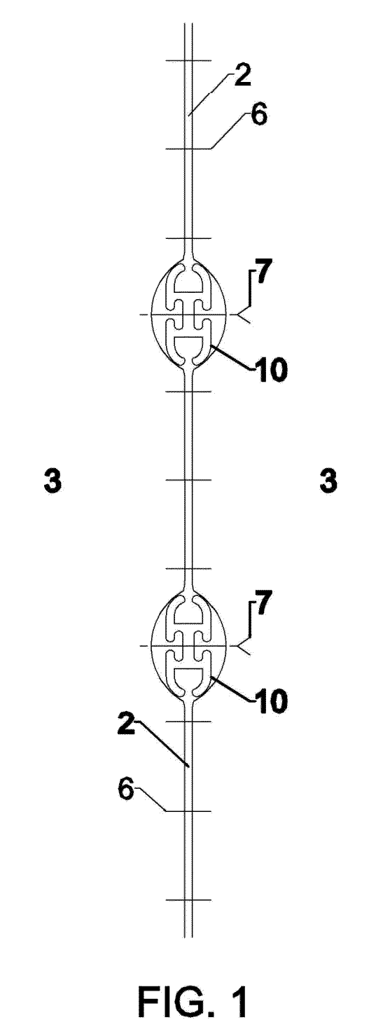

Referring first to FIGS. 1-4D, an exemplifying, non-limiting method for draining and applying negative pressure to a surgical wound 1 in accordance with the invention includes closing a surgical incision 2 by approximating the skin 3 on either side with staples or sutures 6, typically placed in an interrupted manner, but which may be placed as a continuous running suture. The method entails inserting at least one drain 10 between staples or sutures 6 through the incision 2 in the skin 3, perpendicular or nearly perpendicular to the plane of the skin 3, into wound 1 so that an end 11 of each drain 10 is situated above the skin 3 and the opposite end 12 is situated above a closed peritoneal or fascial layer 5 of the wound 1.

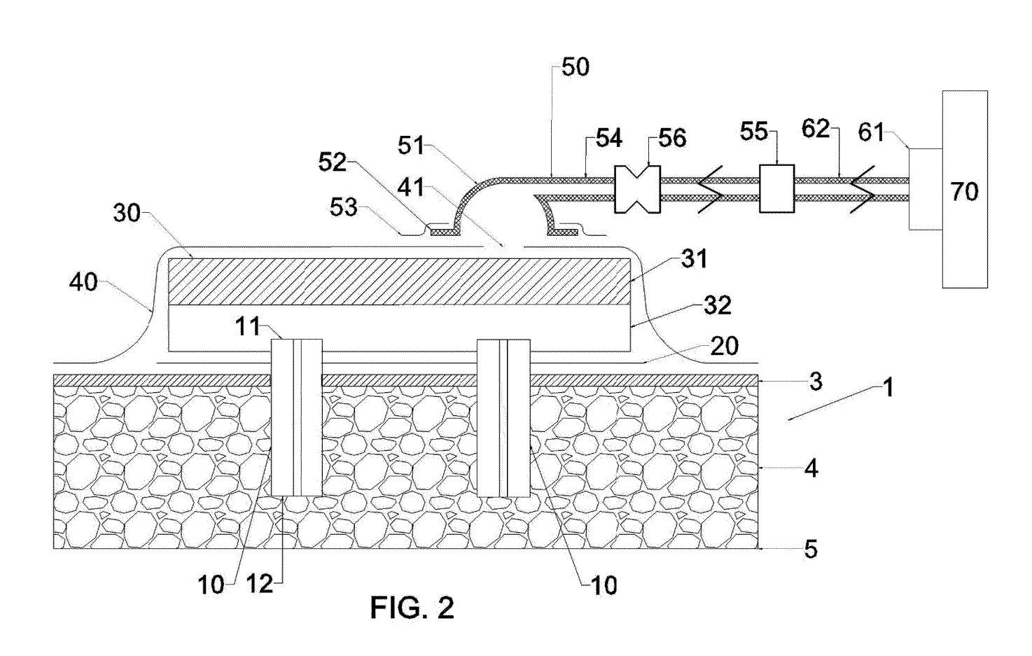

FIGS. 1 and 2 show the use of two drains 10, although the actual number used for each wound draining may vary as determined, e.g., by the surgeon or other operatory personnel. Here drain 10 is shown extending perpendicular to the skin 3 and fascia 5, but in use, a portion of the drain may lie in another orientation, for example, parallel to the fascia as the drain traverses under a skin flap or between tissue planes.

A preferred embodiment of drain 10 is a flat channel drain, a substantially rectangular fluted elastomeric extruded body with multiple longitudinal grooves or channels through which fluid can travel the length of the drain. Thus, at least a portion of the cross-sectional circumference may be open for the entire length of the drain 10. Such a fluted structure is seen in flat Blake surgical drains. A preferred width of flat channel drain 10 is about 7 mm, although about 10 mm drains and drains of various sizes and dimensions may be used. Drains of other structures can be used instead of a flat channel drain 10 as discussed below. Moreover, for the same wound, different types of drains may be used.

Each drain 10 may be secured in this position by a suitable securing mechanism, for example, with a suture tie 7 by passing a suture needle and thread through the skin and drain and tying to form one or more stitches that runs through the skin 3 and the drain 10. Instead of a suture tie 7, other comparable securing mechanisms known to those skilled in this field may be used. Such securing mechanisms are collectively referred to as securing means herein.

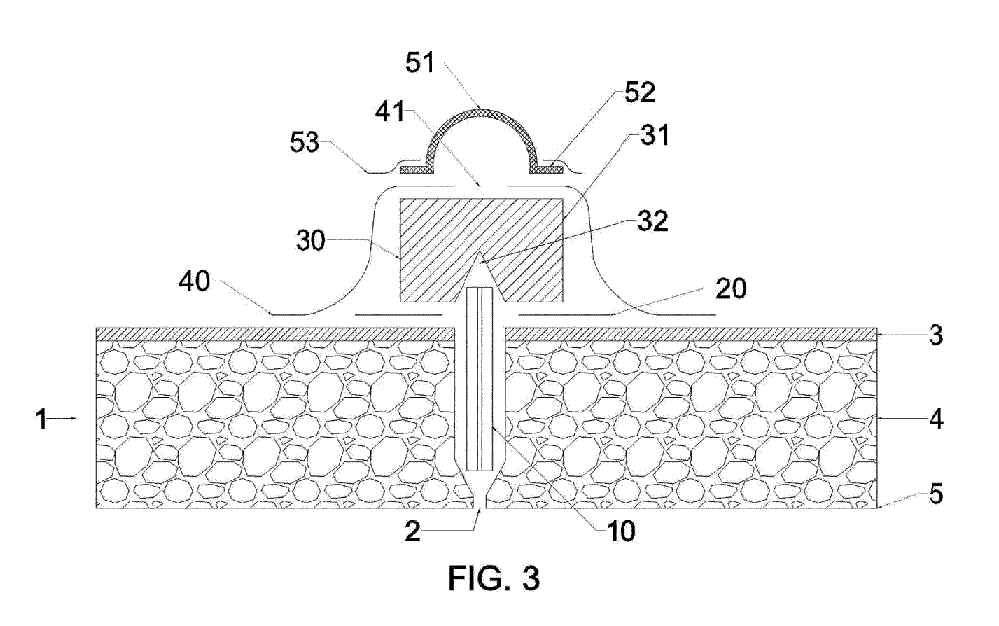

To avoid damage to intact skin by suction to be applied through a sponge 30 that will be placed over the drains 10, protective strips of adhesive film dressing are preferably placed along both sides of the incision 2 for the length of the incision 2 (see FIGS. 2 and 3).

Alternatively, foam sponge 30 may be provided with narrow strips of adhesive film fixed, for example, to its undersurface by adhesive or other technique known in the art. These narrow strips may run the full length of foam sponge 30, or a portion of the length, one strip on either side of the groove, neither strip covering the groove, and be provided with liners, so that when the liners are removed the sponge 30 may be secured in place over the incision and drains, to the skin, with the drains projecting into the groove, by the adhesive film strips.

Adhesive film dressings 20 and the adhesive film strips described in the previous paragraph, serve to protect the underlying skin from damage caused by direct contact with a sponge under negative pressure. The semipermeable nature of the adhesive film dressings and strips allows vapor produced by the skin to traverse the film and be absorbed by the sponge.

An open cell foam sponge 30 is then preferably placed over drains 10 to cover the drains, incision 2 (see FIGS. 2 and 3) and protective strips of adhesive film dressing 20. The sponge 30 would preferably be long enough so it could cover most surgical incisions and in use would be cut to the length of the surgical incision, if necessary. Dimensions of approximately 2 cm thick, 3 cm wide and 40 cm long for the sponge 30 are preferable. The width of one end 31 of the sponge 30 may be expanded (to for example, 5 cm), and possibly assume a substantially circular shape, in order to better accommodate a suction dome 51 and its flange 52. Sponge 30 may contain one or more grooves or slits 32 on its undersurface (FIGS. 2 and 3) into which the drains 10 insert. This allows the sponge 30 to be restrained by the drains 10, so that the sponge 30 stays more readily in place over the incision 2 and drains 10 while an adhesive film drape 40 is applied. Although a single sponge 30 is shown, if necessary, multiple sponges may be used.

The occlusive transparent adhesive film drape 40 with its adhesive surface facing downward, is preferably placed over the sponge 30 to cover the entire sponge 30 and incision 2, and then firmly secured to the healthy skin 3 around the wound margin. The manner in which this securing is performed may be any known to those skilled in the art to which this invention pertains. Coverage of the entire sponge 30 and incision 2 by the adhesive film drape 40 prevents an air leak from developing that would adversely impact the vacuum and suction application. One or more additional adhesive film drapes or dressings may be placed over those described to reinforce all seals.

An aperture 41 is cut into the adhesive film 40 above the sponge 30 (see FIGS. 2 and 3). Onto this aperture 41, a suction dome 51 or other hub of a tube assembly 50 is secured, for example, by means of an adhesive film collar 53 that may be attached to the flange 52 of suction dome 51. Alternatively, a hub of a different design, e.g., a short tube with side perforations or channels, can be inserted on top of or through a portion of the sponge and an adhesive film dressing used to seal an interface between the hub and sponge 30 or tube 54 and sponge 30 so that no air leak would ensue when negative pressure is applied to the sponge 30 through the tube 54. Suction dome 51 is provided preferably integrally attached to tube 54 of tube assembly 50. A connector 55 is provided at the end of tube 54 opposite suction dome 51, that connects to a tube 62 from container 61, either directly or by means of a compatible connector fitting on the end of tube 62. Tube 54 may have a single lumen as shown or multiple lumens in flow communication with suction dome 51 and connector 55. The lumen of tube 54 is placed in flow communication with a suction source 70. A tubing clamp 56 is provided to occlude the tube lumen when circumstances require that tube 54 be separated from the suction source 70, for example, when the patient ambulates.

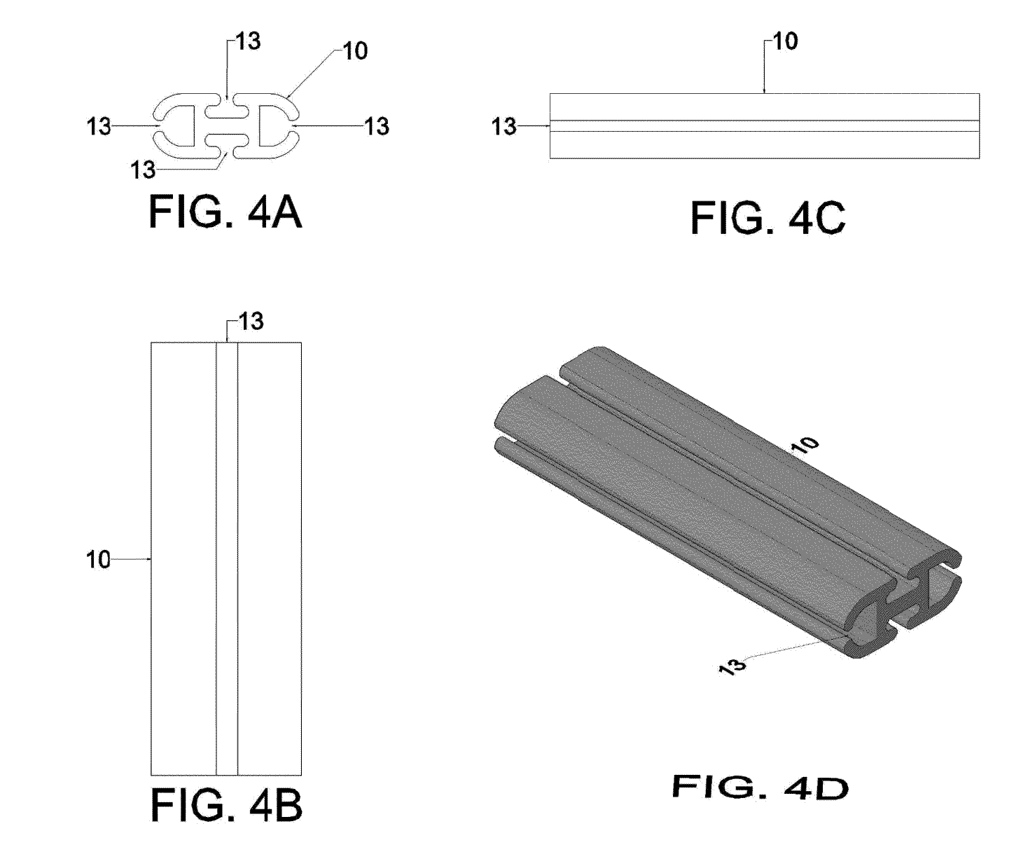

When the suction source 70 is activated, constant or intermittent negative pressure is applied to the sponge 30, each drain 10 and wound 1. Free fluid in the wound 1 is aspirated through channels 13 of each wound drain 10 (see FIGS. 4A-4D), into and through the sponge 30, into the suction dome 51 of tube assembly 50, and through tube 54 and tube 62 into a collection container 61 that is provided with negative pressure by the suction source 70. The adhesive film drape 40 and adhesive film collar 53 prevent the ingress of air and allow a partial vacuum to form within the wound 1, reducing its volume and facilitating the removal of fluid.

Drains 10 may be of varying lengths so as to accommodate varying thicknesses of the fatty subcutaneous layer and wound depths.

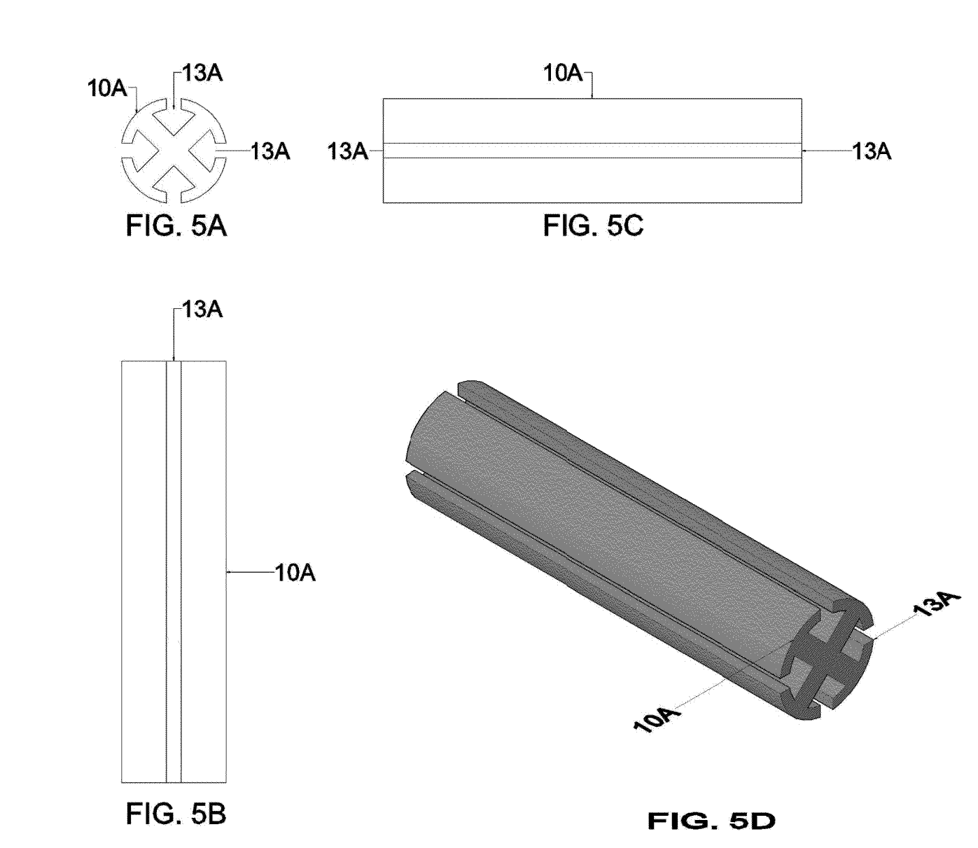

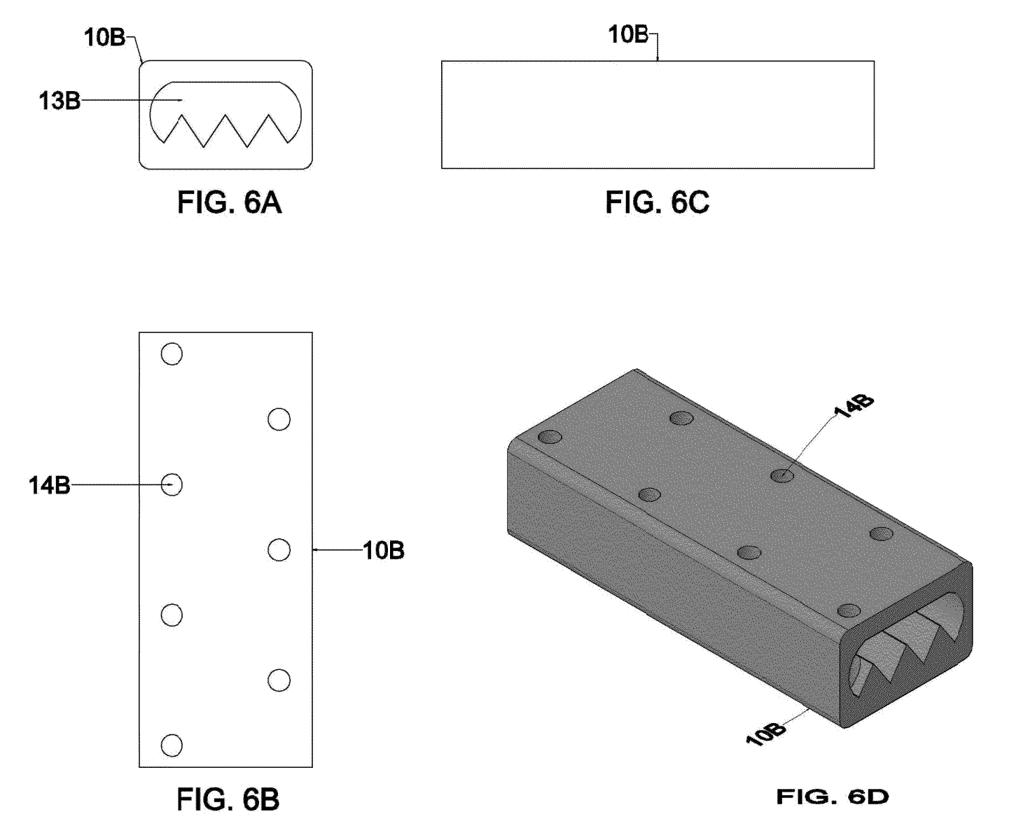

Referring now to FIGS. 5A-6D, round channel drain 10A, having channels 13A and a structure seen in round Blake surgical drains, and rectangular tubular drain 10B with a single lumen 13B and multiple wall apertures 14B, having a structure seen in Jackson Pratt surgical drains, may be used in place of flat channel drain 10 shown in FIGS. 4A-4D. Drain 10A and drain 10B, like drain 10, are structured and have sufficient rigidity so that the drain channels or lumen 13A, 13B do not collapse under negative pressures used in negative pressure wound therapy, and therefore these drains can effectively transmit negative pressure into the wound.

Preferable diameter of the round channel drain 10A and width of flat rectangular tubular drain is about 7 mm, although about 10 mm drains and drains of various dimensions may be used.

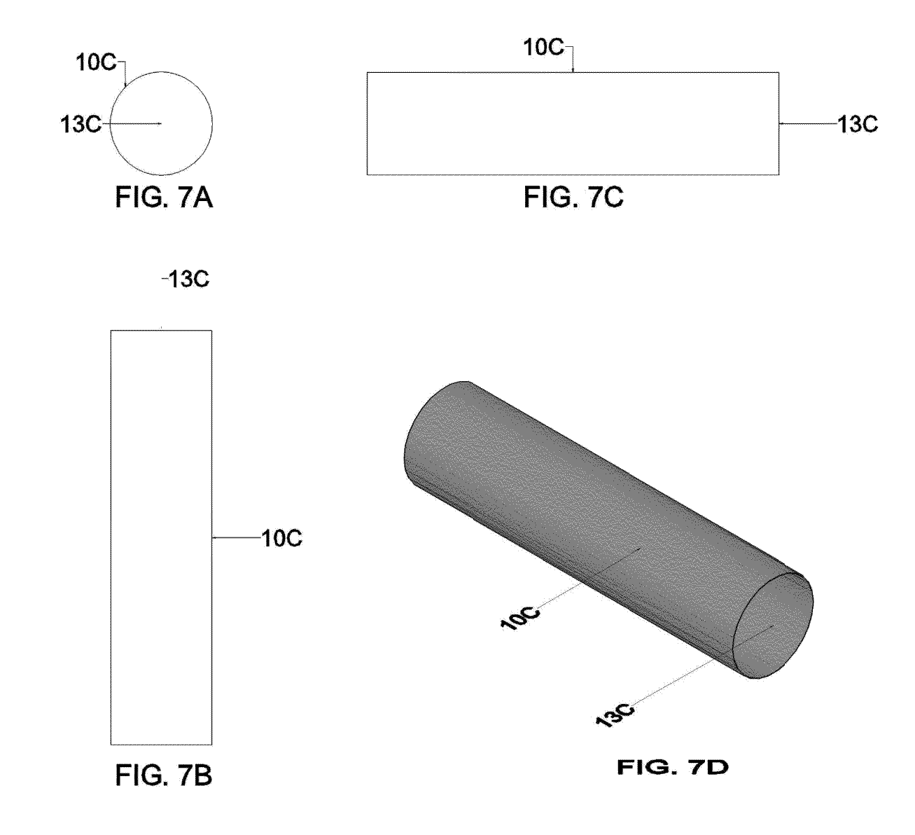

Referring now to FIGS. 7A-7D, a soft thin walled rubber tube 10C having a single lumen 13C, and commonly known as a Penrose drain, is collapsible, and therefore may not as effectively transmit negative pressure into the interior of the wound as effectively as drains 10-10B. Drain 10C can nevertheless be used in place of flat channel drain 10 to provide a conduit for egress of fluids from the wound 1.

The material forming drains 10-10C may be doped with radiopaque material, or a radiopaque material may be co-extruded, molded or woven into the drains, e.g, as a stripe or strand, so that the drains may be more easily detected by radiographic means. This may be helpful in radiographically determining the presence and/or position of the drain within the tissues, and would be helpful in identifying and locating a drain should one accidently submerge beneath the skin without anyone realizing it, especially if the suture 7 holding it dislodged or was inadvertently removed without the drain.

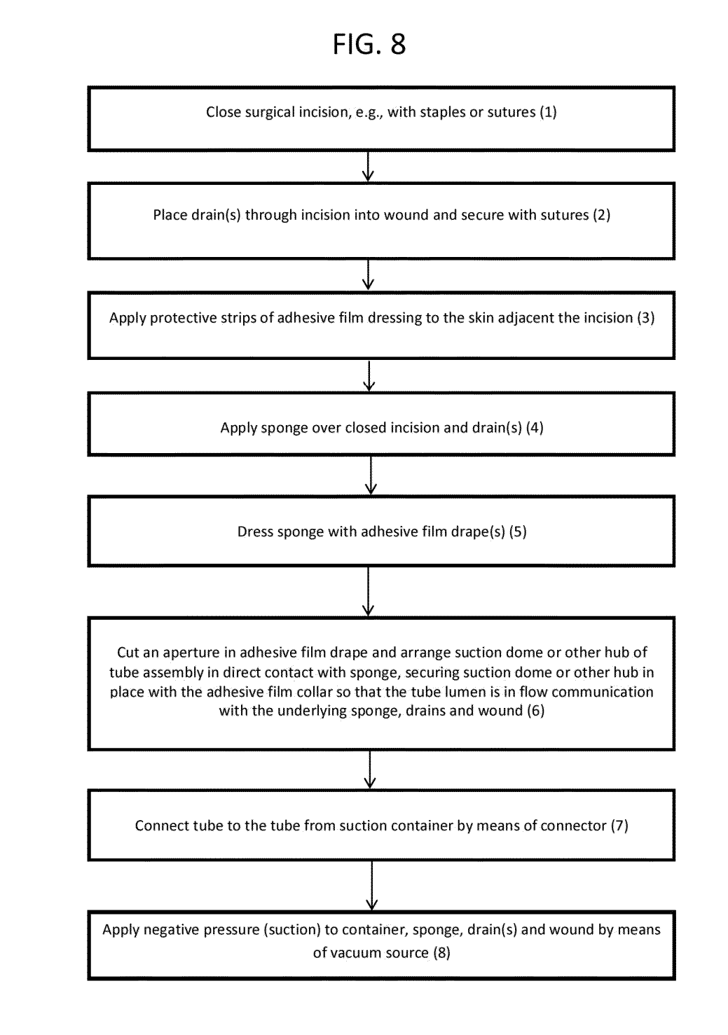

To summarize the basic steps of the method of this invention, which can be seen in chart form in FIG. 8:

Step 1—A surgical incision is closed by approximating skin edges with staples or sutures, or other securing means.

Step 2—One or more drains are placed through the incision into the wound space and secured with sutures. Selection of the number and type of drain is at the discretion of the surgeon, whether one or more of drains 10-10C described above or another comparable drain.

Step 3—Protective strips of adhesive film dressing are applied to the skin adjacent the incision.

Step 4—A sponge is applied over the closed incision and drains.

Step 5—The sponge is dressed with adhesive film drapes, or other comparable means to cover and maintain the sponge in a substantially air-tight manner against the incision and drains.

Step 6—A connector, tube, suction dome or other hub and adhesive film collar assembly is arranged to be in direct contact with the sponge, by cutting an aperture in the adhesive film over the sponge, and securing a suction dome or other hub in place with the adhesive film collar so that the tube lumen is in flow communication with the underlying sponge, drains and wound.

Step 7—The tube is connected to an additional tube from a suction container by means of the connector.

Step 8—Negative pressure is applied to the container, sponge drains and wound by means of a vacuum source.

This order of steps is not an absolute requirement and may be altered. For example, step 6 may be performed before step 5.

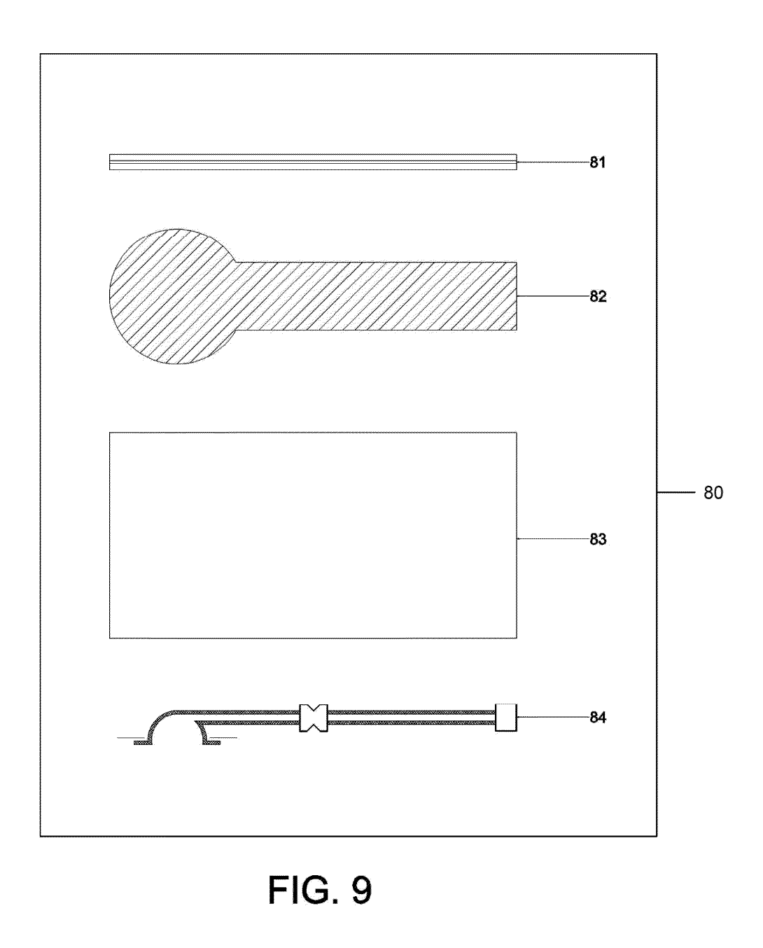

Referring now to FIG. 9, an incisional wound dressing kit 80 may be provided containing components necessary to dress a wound in accordance with this method in order to facilitate the performance of the method of this invention. This kit 80 may contain a first component 81 comprising multiple drains 10 cut to a specific or varying lengths, or a single length of an elastomeric body that may be cut by the surgeon into multiple drains of lengths that the surgeon determines to be optimal at the time of surgery, depending upon the depth of the patient's wound 1 to be drained. That is, the first component 81 represents either a single drain, or a body that can be cut into multiple drains. In either case, a plurality of first components 81 may be included in the kit 80. The kit 80 may contain a second component 82 being a length of open cell foam, possibly containing expanded portion 31 and slit 32, to be used as the sponge 30 for placement over the incision 2 and drains 10. The kit 80 may contain a third component 83, strips and or sheets of adhesive film, together with their liners, to be used as dressings 20 and drapes 40. A fourth component 84 of such a wound dressing kit 80 may include a tube assembly 50 comprising tube 54 to transmit negative pressure to the sponge 30 and wound 1 and through which fluid is drawn from the wound, a suction dome 51 or other hub that conveniently adapts to the sponge 30 so that the sponge is in flow communication with the interior of tube 54, and adhesive film collar 53 and liners that are used to secure and seal the suction dome 51 to the sponge 30 so that air ingress is prevented at the suction dome and sponge interface. The tube assembly 50 may also include a connector 55 on the end of the tube opposite the suction dome 51 that allows the tube 54 to connect with a tube 62 from the container 61 that is used to collect fluid from the wound, and which is supplied with negative pressure by suction source 70. Instructions for use of the contents of the kit 80 may also be provided with the kit 80.

While the apparatus and methods of this invention may be used at the time of the surgical operation, the same apparatus and methods may be used in the postoperative period to drain the wound if a wound infection or seroma develops or is suspected.

With the foregoing apparatus and method, several objectives and advantages are provided. Among others, the present invention provides apparatus and methods for expediting the healing of a surgical wound, provides apparatus and methods for reducing the chance of developing a wound infection, provides apparatus and methods for reducing the labor required for the postoperative care of a surgical wound, and/or provides apparatus and methods to reduce the cost of postoperative care of a surgical wound.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

1. A method of wound management, comprising:
placing at least one drain into a closed incisional wound in a position in which it traverses skin;
placing a sponge over the closed incisional wound and the at least one drain to cause the at least one drain to be in flow communication with the sponge; and
applying negative pressure to the closed incisional wound via the sponge overlying the closed incision and thus to the at least one drain in flow communication with the sponge.

2. The method of claim 1, wherein the step of placing at least one drain into the wound comprises placing a plurality of drains along the length of the wound.

3. The method of claim 1, further comprising maintaining the applied negative pressure by surrounding the sponge and wound with at least one adhesive film drape.

4. The method of claim 1, wherein the step of applying negative pressure to the wound comprises connecting at least one tube between a vacuum source and the sponge.

5. The method of claim 4, further comprising interposing a hub between the tube in flow communication with the vacuum source and the sponge.

6. The method of claim 5, further comprising maintaining the applied negative pressure by surrounding the sponge and wound with at least one first adhesive film drape; and
securing the hub over an aperture in the at least one first adhesive film drape over the sponge using a second adhesive film drape.

7. The method of claim 6, where the hub is a suction dome and the second adhesive film drape is an adhesive film collar.

8. The method of claim 4, further comprising interposing a container between the sponge and vacuum source to collect wound drainage.

9. The method of claim 1, further comprising configuring the at least one drain to define an open conduit that remains open when negative pressure is applied to its interior.

10. The method of claim 1, further comprising configuring the at least one drain as an elastomeric channel drain, wherein at least a portion of the cross-sectional circumference is open for the entire length of the drain.

11. The method of claim 1, further comprising configuring the at least one drain as a flat channel drain.

12. The method of claim 1, further comprising configuring the at least one drain as an elastomeric tubular drain.

13. The method of claim 1, further comprising securing the drain to the skin with at least one stitch that runs through the skin and the drain.

14. The method of claim 1, wherein the sponge is provided with a longitudinal groove arranged along its undersurface, the step of placing the sponge over the wound and the at least one drain comprising positioning the sponge such that an end of each of the at least one drain is situated in the groove.

15. A kit of materials to dress a wound, comprising:
at least one elastomeric body that may be cut into multiple drains, said body including at least one longitudinally extending channel such that each of said drains includes a channel through which fluid flows when said drain is placed into the wound in a position in which it traverses skin,
an open cell sponge adapted to overlie said drains and the wound,
at least one adhesive film drape with a liner configured to overlie said sponge when said sponge overlies the wound and said drains,
a tube assembly comprising a hub that adapts to the sponge and a tube extending from the hub that has a lumen adapted to connected to a vacuum source, whereby negative pressure applied from the vacuum source is transmitted through said tube assembly to said sponge through said drains into the wound.

16. The kit of claim 15, wherein said hub is a suction dome.

17. The kit of claim 15, wherein said hub has a flange with an attached adhesive film collar and liners for adapting said hub to said sponge

18. The kit of claim 17, further comprising a connector that connects the tube to an additional tube from a container which transmits negative pressure from the vacuum source, and a clamp that reversably occludes said tube of said tube assembly.

19. The kit of claim 17, wherein said sponge has a widened end to accommodate said hub.

20. The kit of claim 15, wherein an undersurface of said sponge contains at least one longitudinal grove.

21. An arrangement of wound management, comprising:
at least one drain each adapted to be placed into a closed incisional wound in a position in which it traverses skin;
a sponge adapted to be placed over the closed incisional wound and said at least one drain to cause said at least one drain to be in flow communication with said sponge,
a system for applying negative pressure to the closed incisional wound directly via said sponge overlying the closed incision and thus indirectly to said at least one drain in flow communication with said sponge via said sponge.
